(19) United States
(12) Patent Application Publication
(10) Pub. No.: US 2026/0002201 A1
(43) Pub. Date: Jan. 1, 2026

(54) Apparatus and method for testing of a biological or environmental sample

(71) Applicant: ZIP DIAGNOSTICS PTY LTD, Collingwood, Victoria (AU)

(72) Inventors: William Robb HOPPER, East Ivanhoe, Victoria (AU); Jonathan Scott RICHARDS, Fitzroy, Victoria (AU); Hanh Hong Thi NGUYEN, Glenroy, Victoria (AU)

(21) Appl. No.: 18/881,706

(22) PCT Filed: Jul. 5, 2023
(86) PCT No.: PCT/AU2023/050623

§ 371 Date: Jan. 6, 2025

(30) Foreign Application Priority Data
Jul. 7, 2022 (AU) 2022901908

(51) Int. Cl. C12Q 1/6844; C12Q 1/70; G01N 21/64; G01N 21/76; G01N 21/78

(52) U.S. Cl. CPC C12Q 1/6844; C12Q 1/701; G01N 21/6428; G01N 21/763; G01N 21/78

(57) ABSTRACT

An apparatus for testing of a biological or environmental sample is disclosed. The apparatus includes: a vessel defining an interior chamber and an opening to the chamber, with the opening being the only means of entry and exit from the chamber; one or more dried beads disposed within the chamber, and wherein the opening is configured to allow a fluid containing the biological or environmental sample to enter the chamber for testing of a reaction with the one or more dried beads therein; and a retaining component disposed between the opening and the one or more dried beads within the chamber; and wherein the retaining component is configured to prevent the one or more dried beads from exiting the vessel through the opening when open, and to allow fluid in the vessel to flow past and/or through the retaining component to interact with the one or more dried beads therein.

### TECHNICAL FIELD

The present disclosure relates to diagnostic and biomedical tests involving biological or environmental samples. More specifically, the present disclosure relates to a container or apparatus for testing of a biological or environmental sample.

### BACKGROUND

Biological samples (e.g., urine, saliva, blood and other bodily fluids) and environmental samples (e.g., water collected from lakes, reservoirs, aquifers or streams) are often used in biological tests for detecting the presence or absence of one or more protein or nucleic acid target(s). Biological sample types can be in either liquid or solid form. For example, liquid samples can be added directly, or in a diluted form, to a test workflow. Similarly, solid tissue samples or swab samples can be collected and then eluted into a liquid form as part of a sample preparation workflow and then used for testing procedures.

For point-of-care testing and laboratory testing, test reagents are often provided as spherical or bead shaped lyophilised or other dried materials which are deposited in a tube or a well of a plate. The process of lyophilisation produces a stable preparation by rapid freezing and dehydration of the material under high vacuum. Lyophilised or other dried materials are stable at ambient temperatures, allowing point-of-care use without the need for refrigeration infrastructure. For example, polymerase chain reaction mixtures (PCR master mix) represent a batch of test reagents that are at optimal concentrations within a PCR tube or 96-well plate and can be deposited within tubes as bead shaped lyophilised materials. The lyophilised bead materials can include DNA polymerase, dNTPs, MgCl2, buffers and oligonucleotide primers/probes that bind to target nucleic acids. Typically, these lyophilised bead materials can be reconstituted with PCR-grade water or diluted samples. The inclusion of test reagents in the form of lyophilised beads reduces pipetting, risk of contamination, is convenient, saves time and reduces errors associated with mixing. Additionally, lyophilised bead materials can be stored for months at room temperature, making them ideal for field work where accessibility and efficiency are needed.

A number of challenges arise when opening test tubes with lyophilised or freeze-dried beads. Lyophilised beads are manufactured in the form of rounded beads that contain distinct reagents and are deposited in tubes. While inside the tube, the lyophilised beads are prone to becoming electrostatically charged, which can cause them to exit the tube rapidly upon opening by the user, as illustrated in FIG. 1A. Alternatively, the electrostatically charged lyophilised beads can become stuck on the top of the tube where they can become contaminated by the user or lost to the outside environment. The loss of the material may result in the test material having to be replaced, or the reaction being performed under non-ideal conditions. The problem can be exacerbated by the inclusion of magnetic (steel) mixing beads, which can also become electrostatically charged within the tube. Current solutions to this problem include the incorporation of bead retainers in the form of filters or tetrahedral parts, as illustrated in FIG. 1B. However, these small parts are difficult to manufacture, can capture some of the lyophilised test material, and may lead to difficulty in mixing, which can cause variation or error when measuring the test. Additionally, depending on its composition, the retaining object can interfere with the assay reaction or readout.

It is desired to overcome or alleviate one or more difficulties of the prior art, or to at least provide a useful alternative.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

Some embodiments relate to an apparatus for testing of a biological or environmental sample, the apparatus including:
a vessel defining an interior chamber and an opening to the chamber, with the opening being the only means of entry and exit from the chamber;
one or more dried beads disposed within the chamber, and wherein the opening is configured to allow a fluid containing the biological or environmental sample to enter the chamber for testing of a reaction with the one or more dried beads therein; and
a retaining component disposed between the opening and the one or more dried beads within the chamber; and
wherein the retaining component is configured to prevent the one or more dried beads from exiting the vessel through the opening when open, and to allow fluid in the vessel to flow past and/or through the retaining component to interact with the one or more dried beads therein.

The vessel may be transparent to facilitate visualisation of at least one of: (i) the one or more dried beads; (ii) the sample; and/or (iii) the retaining component when disposed within the chamber.

The vessel may comprise a cap configured to seal the opening. The cap may be a screw cap comprising cap threads configured to engage with a corresponding thread on the vessel. The cap and the vessel may be integrally formed. The cap may be a flip-top cap.

The one or more dried beads may include diagnostic reagents. The one or more dried beads may include one or more polymerase chain reaction reagents. The one or more dried beads may include one or more oligonucleotide primers.

The one or more dried beads may comprise a first lyophilised primer bead containing a first lyophilised primer, wherein the first lyophilised primer is configured to be reconstituted by the fluid to interact with a first target nucleic acid. The first lyophilised primer bead may comprise at least one of the one or more polymerase chain reaction reagents. The one or more dried beads may comprise a second lyophilised primer bead containing a second lyophilised primer, wherein the second lyophilised primer is configured to be reconstituted by the fluid to interact with a second target nucleic acid. The one or more dried beads may comprise three or more lyophilised primer beads.

The one or more dried beads may comprise a lyophilised probe quencher bead. The one or more dried beads may comprise a lyophilised enzyme bead. The one or more dried beads may comprise a lyophilised deoxynucleotide triphosphate (dNTP) bead.

Each of the lyophilised beads may comprise: (i) an excipient; and/or (ii) betaine.

The apparatus may further include a magnetic mixing component. The magnetic mixing component may be composed of steel.

The retaining component may be approximately spherical. The retaining component is not a filter. An amount of reagent or primer in the fluid may be unchanged as it passes through or around the retaining component during interaction with the sample in the fluid. The retaining component may be configured for an amount of the fluid exiting the retaining component to be the same as an amount of the fluid that entered the retaining component. The retaining component may be configured to: (i) float on top of the fluid; (ii) be partly immersed in the fluid; or (iii) be fully immersed in the fluid.

The retaining component may comprise a hub defining at least one passage extending between opposed first and second surfaces of the retaining component and configured to allow the fluid to enter and exit the retaining component through the at least one passage, wherein the first surface faces the opening of the vessel, and the second surface faces the one or more dried beads within the chamber of the vessel. The retaining component may comprise a prong extending from the first surface of the retaining component.

A diameter of the at least one passage may be smaller than a diameter of the one or more dried beads to prevent the one or more dried beads from passing through the passage. The retaining component may comprise a plurality of outwardly facing side surfaces, and at least one recessed surface, wherein the recessed surface is recessed relative to at least one of the outwardly facing side surfaces so as to be positioned away from an interior wall of the vessel. The plurality of outwardly facing side surfaces may comprise a contact surface configured to engage the interior wall of the vessel with a press fit to secure the retaining component in the chamber.

The retaining component may be composed of a composite plastic. The retaining component may be composed of polypropylene. The retaining component may be composed of polystyrene. The retaining component may be composed of a wax material. The retaining component may be composed of a wax-based material.

The retaining component may be a polypropylene sphere comprising a shell defining: (i) a hollow; and (ii) at least one aperture in the shell in communication with the hollow, wherein the aperture is configured to allow the fluid to enter and exit the retaining component through the shell. The hollow may be filled with a wax or wax-based material.

Some embodiments relate to a diagnostic test kit including:
the apparatus of any one of the preceding claims, and
lysis buffer.

Some embodiments relate to a diagnostic test system comprising:
the diagnostic test kit as described herein;
a test instrument configured to generate a movable magnetic field, wherein movement of the magnetic field causes a corresponding movement of a magnetic mixing component within the apparatus to mix contents within a vessel of the apparatus.

Some embodiments relate to a test method, including:
introducing a fluid containing a biological or environmental sample into the apparatus as described herein.

The apparatus may include a magnetic mixing component, and the method may further include moving the magnetic mixing component to the top of the apparatus to facilitate reading of a test result. The reading of the test result may be by a colorimetric, fluorescence or bioluminescent instrument. The test method may comprise applying a movable magnetic field to the magnetic mixing bead, wherein movement of the movable magnetic field causes corresponding movement of the magnetic mixing component within the vessel of the apparatus.

Some embodiments relate to a method of manufacturing lyophilised beads, wherein the lyophilised beads are subjected to quality control (QC) tests comprising at least one of: (i) counting the beads; (ii) checking sizes of the beads; and (iii) checking bead colours if visual dyes are used to colour the beads.

In one aspect, the present disclosure provides a container for testing of a biological or environmental sample, the container including:
a vessel with one or more dried beads disposed therein and having an opening configured to receive a biological or environmental sample for testing therein; and
a retaining component disposed above the one or more dried beads within the vessel, and configured to prevent the dried beads from exiting the vessel when open;
wherein the retaining component is at least approximately spherical and configured to prevent the one or more dried beads from exiting the vessel when open, and to allow fluid added to the vessel to flow past the retaining component to interact with the dried beads therein.

In another aspect, the present disclosure provides a diagnostic test kit including:
the container of any one of the preceding embodiments, and
instructions for use of the diagnostic test kit.

### BRIEF DESCRIPTION OF THE DRAWINGS

Some embodiments of the present disclosure are hereinafter described, by way of example only, with reference to the accompanying drawings, wherein:

FIGS. 1A and 1B are cross-sectional side views of prior art tubes containing dried beads and a magnetic mixing bead, illustrating (FIG. 1A) the opening of a tube without any retaining component, and (FIG. 1B) three examples labelled (i), (ii), (iii) of prior art retaining components.

FIG. 2 is a sequence of schematic side views of a container or an apparatus for testing of a biological or environmental sample in accordance with an embodiment of the present disclosure and illustrating a method for adding fluid to the container or apparatus and mixing of its contents with a magnetic mixing bead.

FIGS. 3A to 3D are images illustrating addition of fluid to a container or apparatus in the form of a tube containing dried beads, a magnetic mixing bead and a spherical retainer or retaining component according to an embodiment of the present disclosure, where FIG. 3A is a diagram of the tube closed by a cap, FIG. 3B is a diagram of the tube open without the cap, and FIGS. 3C and 3D are diagrams of the open tube with fluid added. FIG. 3C is a diagram of the open tube with the retaining component fully submerged in the fluid, whereas FIG. 3D is a diagram of the open tube with the retaining component only partly covered by the fluid.

FIGS. 4A-4C are views showing an embodiment of the retainer or retaining component. FIG. 4A is a perspective view of the retainer or retaining component, and FIG. 4B is a perspective view of the retainer or retaining component of FIG. 4A within a vessel. FIG. 4C is a cross-section view of the vessel of FIG. 4B showing the retainer or retaining component therein.

FIGS. 5A to 5E are cross-section side views of a container or apparatus containing dried beads, magnetic mixing beads and a retaining component in accordance with some embodiments of the present disclosure, including (FIG. 5A) a small retaining component, (FIG. 5B) a large retaining component, (FIG. 5C) a small retaining component in a screw cap tube, (FIG. 5D) a small retaining component in a flat-bottom tube, (FIG. 5E) a small retaining component in a small PCR tube.

FIGS. 6A, 6B and 6C are cross-sectional (FIG. 6A) side and (FIGS. 6B and 6C) top views of a vessel containing dried beads, magnetic mixing beads and a retaining component in accordance with embodiments of the present disclosure, and including projections to support the retaining component and prevent it from falling to the bottom of the vessel. FIG. 6B shows the vessel with the retaining component supported on the projections, and FIG. 6C shows the vessel without the retaining component.

FIG. 7 is a side view of a dual tube cartridge with dried beads, magnetic mixing beads and a retaining component in accordance with an embodiment of the present disclosure.

FIG. 8 is a sequence of schematic diagrams illustrating a method for test assay using containers or apparatuses containing dried beads, magnetic beads and several retainers in accordance with some embodiments of the present disclosure.

### DETAILED DESCRIPTION

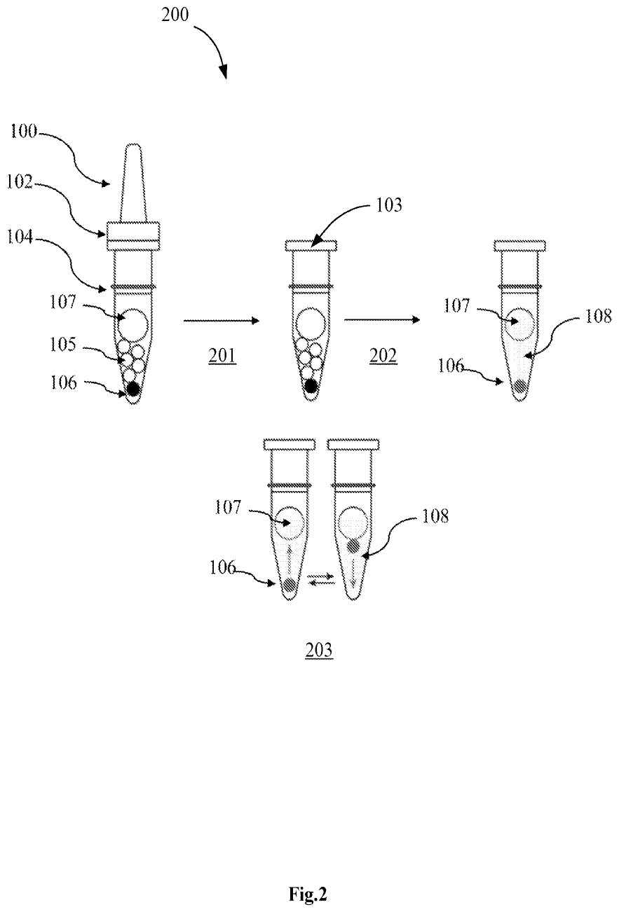

Embodiments of the present disclosure include a container or apparatus for testing of a biological or environmental sample. Referring to FIG. 2, a container or apparatus 100 for testing of a biological or environmental sample, according to a testing method 200, includes a cap component 102 configured to cover an opening 103 of a vessel 104. The vessel 104 defines an interior chamber and has one open end defining an opening 103 to the chamber. The vessel 104 may come with one or more dried beads 105 disposed therein and the opening 103 may be configured to receive a biological or environmental sample for testing therein. Specifically, the sample may react with one or more (or none) of the beads 105 within the chamber, and the reaction may be analysed as part of a test for determining the presence of an analyte in the sample. The vessel 104, along with the cap component 102, ensures proper containment of the sample during testing. The cap component 102 is designed with a secure sealing mechanism to prevent any leakage or contamination of the sample during testing and transportation. This could involve the use of O-rings, gaskets, or other sealing methods. The cap component can either be a screw-on cap that provides a threaded mechanism for securing sealing the opening of the vessel. Alternatively, the cap component could be a snap-on cap that utilises a locking mechanism to seal the opening of the vessel.

In certain embodiments, heat can be applied to initiate or expedite the reaction between the beads 105 and the analyte present in the sample. The heat may “amplify” the sample at a specific temperature, called the amplification temperature. The heat can be applied in a controlled or gradual manner to ensure optimal reaction conditions. Temperature control systems such as thermoelectric modules or Peltier cells can be used to achieve and maintain the desired amplification temperature. These can activity monitor and adjust the temperature within the vessel to ensure precise control during the testing process. A suitable amplification temperature can be around 65° C. (degrees Celsius). Some reactions may require a variation in the amplification temperature of 1-2° C. Examples of suitable amplification temperature ranges include temperatures of around 60° C. to 70° C., around 65° C. to 70° C., around 60° C. to 65° C., around 63° C. to 67° C., around 65° C. to 67° C., or around 63° C. to 65° C. The specific amplification temperature ranges provided offer flexibility in accommodating various test requirements and ensure reliability and sensitivity in results.

The opening 103 to the vessel 104 is configured to be the only means of entry to and exit from the chamber. Primarily, the opening 103 remains covered and sealed by a cap component 102 during transport or storage, and in this configuration the vessel 104 can be described as being in a closed state, or simply ‘closed’. However, to introduce a sample into the vessel 104 for testing purposes, as outlined in testing method 200 (discussed below), the vessel 104 must be opened to access its interior chamber containing the beads 105. This is achieved by removing the cap component 102, thus transitioning the vessel 104 into an ‘open’ state, or simply ‘open’. In addition, the container or apparatus 100 may comprise a mixing component 106 is disposed within the chamber of the vessel 104. The mixing component 106 may be configured to move the beads 105 within the vessel 104, ensuring efficient mixing and interaction with the sample during testing. The mixing component 106 may be non-magnetic or comprise non-magnetic portions. In some embodiments, the mixing component 106 is magnetic.

A retaining component 107 is disposed among the one or more dried beads 105 within the vessel 104, and is configured to prevent the dried beads 105 from exiting the vessel 104 when open. The retaining component 107 may be located between the beads 105 and the opening 103 of the vessel 104. In some embodiments, particularly involving cap component 102 and/or vessel 104 made from plastic, the dried beads 105 are prone to static electricity, which could cause them to leap out of the vessel 104 if the retaining component is not present. It should be understood that this prevention is in the context of the container or apparatus in normal use, where the open vessel 104 is generally in an upright position and not subjected to inversion, or vigorous agitation.

In some embodiments, the retaining component 107 is at least approximately spherical in shape. This shape is configured to not only prevent the one or more dried beads 105 from exiting the vessel 104 when it is open, but also to allow fluid to be added to the vessel to interact with the dried beads 105 therein. The retaining component is not a filter, which may cause the undesirable capture of some of the lyophilised test material before or after it is reconstituted. In some embodiments, the amount of test material in the fluid is unchanged as it passes around (or in some embodiments, through) the retaining component. In some embodiments, an amount of reagent or primer in the fluid is unchanged as it passes through or around the retaining component.

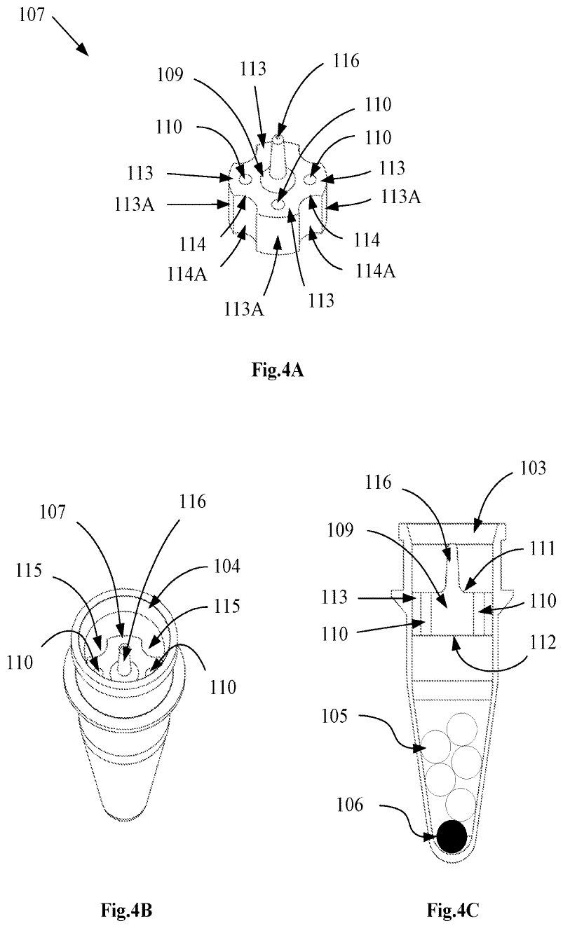

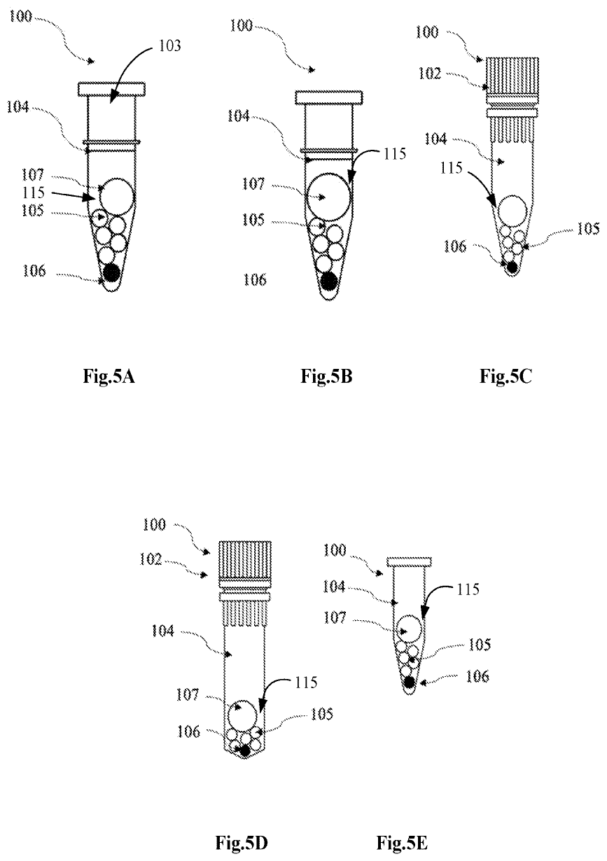

In some embodiments, such as shown in FIGS. 4A-4C, the retaining component 107 has a non-spherical shape, but is still configured to prevent inadvertent exit of the one or more dried beads 105 while allowing fluid to be added to the vessel 104 to interact with the dried beads 105 therein. Embodiments of the retaining component 107 will be subsequently discussed in more detail below.

Turning again to FIG. 2, in some embodiments, the container or apparatus 100 further comprises a magnetic mixing component 106. The magnetic mixing component 106 is configured to be placed within the vessel 104 and positioned among the dried beads 105. When the retaining component 107 is in placed within the vessel 104, both the dried beads 105 and the mixing component 106 are allowed to freely move within the chamber. For example, mechanical force (such as shaking of the vessel 104) or a magnetic field could be employed to initiate movement of the mixing component 106, which in turn agitates the surrounding dried beads 105, facilitating their interaction with the sample. The magnetic field may be generated by an external permanent magnet that is moved in proximity of the vessel 104 to stimulate the movement of the magnetic mixing component 106. The inventors have identified that the mechanical agitation provided by the movement of the mixing component 106 improves reaction time and consistency. The movement by the mixing component 106 is not impeded or restricted by the retaining component, which allows for unobstructed mixing.

FIG. 2 shows a container or apparatus 100 for testing of a biological or environmental sample according to an embodiment of the present disclosure. The container or apparatus 100 comprises the vessel 104, within which a retaining component 107 is disposed. The retaining component 107 is arranged to be between the opening 103 and the one or more dried beads 105 within the vessel chamber. The retaining component 107 thereby impede movement of the one or more dried beads 105 towards the opening 103, and may prevent the beads 105 from exiting the chamber through the opening 103. For example, the retaining component 107 may be disposed above the one or more dried beads 105 and the magnetic mixing component 106.

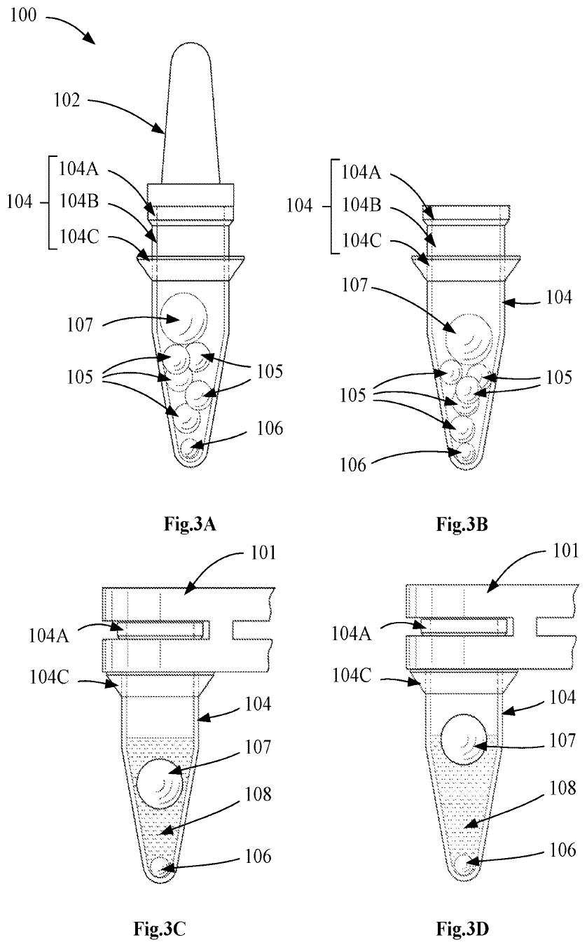

The vessel 104 is sealed with a cap 102 (also shown in FIG. 3A). Further in FIG. 2, when the cap component 102 is removed (such as when the cap 102 is removed at 201 of method 200) and the vessel 104 is open and upright (also shown in FIG. 3B), the retaining component 107 within the vessel 104 prevents the dried beads 105 from exiting the vessel 104. Upon the addition of fluid 108 into the vessel 104, as at 202 of method 200 (also shown in FIG. 3C), the retaining component 107 permits the fluid 108 to pass and contact the beads 105. For example, in some embodiments, the retaining component 107 moves or shifts (or floats) to the side of the vessel 104, thereby creating a gap through which fluid 108 can flow and which allows the one or more dried beads 105 to be dissolved and the dried compounds in the beads 105 to be reconstituted. Further, when the vessel 104 is input into a test device or instrument, a magnetic field can be applied to the magnetic mixing component 106 to facilitate mixing of the fluid 108, as at 203 of method 200. After the addition of the fluid 108, the cap 102 may be reattached to prevent the biological material from escaping or contaminants entering during the testing process. The retaining component 107, which in some embodiments may be spherical in shape, can thereby move aside without interfering with the magnetic mixing component 106, during the mixing operation at 203. The retaining component 107 does not inhibit or prevent the mixing operation at 203, but remains in direct contact with the fluid within the vessel 104. The retaining component 107 may be better able to move out of the way of the magnetic mixing component 106 and remain in contact with the fluid 108 where the retaining component 107 is at least approximately spherical. The spherical retaining component 107 can smoothly roll or shift to the side, making way for the fluid 108 to interact with the dried beads 105. Additionally, the spherical retaining component 107 can move in all directions allowing it to move out of the way, regardless of the direction of the fluid or magnetic force applied.

FIGS. 3A-3C show prototypes of an embodiment of the container or apparatus 100. As shown in FIG. 3A, a container or apparatus 100 for testing of a biological or environmental sample includes a vessel 104 closed and sealed with a cap 102, and containing a retaining component 107, dried beads 105, and a magnetic mixing component 106. The vessel 104 may comprise a lip 104A which defines the opening to the chamber of the vessel 104 and is configured to engage with the cap 102 to cover the opening. The vessel 104 may further comprise a neck region 104B that is defined between a shoulder 104C and the lip 104A. The retaining component 107 is configured to prevent the one or more dried beads 105 from exiting the vessel 104 when open, as shown in FIG. 3B and to allow the fluid 108 to be added to the vessel 104 to interact with the dried beads 105 therein, as shown in FIG. 3C. The retention of the one or more dried beads 105 by the retaining component 107 is primarily achieved by the positioning of the retaining component 107 above the one or more dried beads 105, by the weight of the retaining component 107, and the gap formed between the inner side wall of the vessel 104 and the retaining component 107, being smaller than the size of the one or more dried beads 105.

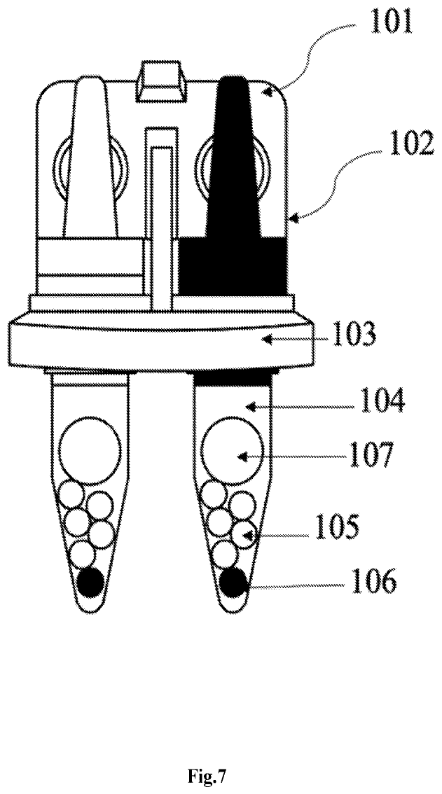

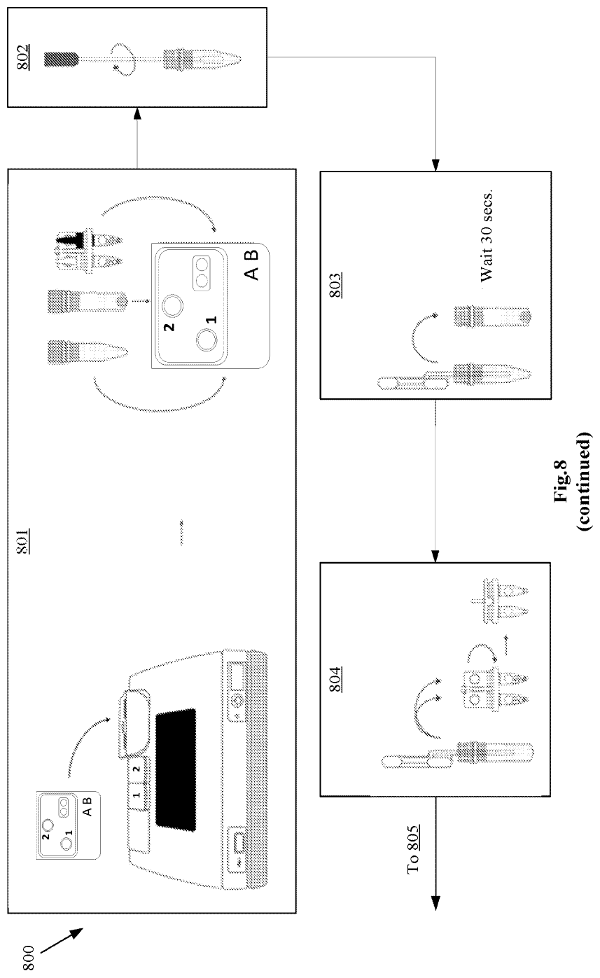

FIG. 3B shows the vessel 104 in an open configuration, with the cap 102 removed. FIG. 3B shows the retaining bead 107 sitting on top of the dried beads 105, and sized to prevent the beads 105 passing around the retaining bead 107. The user is then able to add the fluid 108 (for example, lysis buffer containing the sample to be tested) into the vessel 104, thereby dissolving the beads 105 and releasing (and reconstituting) the active component(s) of the one or more dried beads 105, such as shown in FIG. 3C and FIG. 3D. FIGS. 3C and 3D (with additional reference to FIG. 7) show the container or apparatus 100 received in a cartridge 101 which may be configured to hold one or more of the apparatuses or containers 100. In some embodiments, the cartridge 101 is configured to hold the container or apparatus 100 by the neck region 104B. The shoulder 104C and the lip 104A may protrude from the wall of the vessel 104 to facilitate engagement with and retention by the cartridge 101. Once the one or more dried beads 105 are fully dissolved in the fluid 108, the retaining bead 107 may be fully submerged in the fluid 108, such as shown in FIG. 3C. Once the beads 105 are fully dissolved in the fluid 108, the retaining bead 107 may float on top of the fluid 108, wherein all or the majority of the retaining bead 107 is above the surface of the fluid 108. In some embodiments, the retaining bead 107 is partially submerged in the fluid 108, such as shown in FIG. 3D. Approximately half of the retaining bead 107 may be below the surface of the fluid 108 so that there is more space for the magnetic mixing component 106 to move in the chamber. Having the retaining component 107 floating on top of the fluid 108, or partly below the surface of the fluid 108, may have advantages in limiting evaporation of the fluid 108.

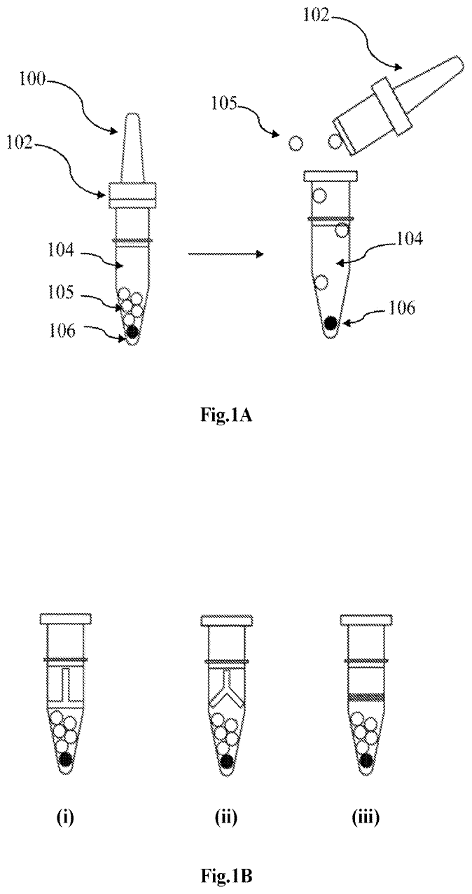

In the embodiment shown in FIGS. 3A-3C, the retaining component 107 is a ball, or is at least substantially spherical. As the fluid is added to one side of the vessel 104, the retaining component 107 moves to the other side of the vessel 104 and rotates, thereby washing any dried bead material that may have stuck to the retaining component 107. The approximately spherical form of the retaining component 107 is advantageous over prior art retaining components (e.g., as shown in FIG. 1B) as it is able to move freely in the fluid and not trap lyophilised material, which can affect the measurement or mixing of the sample.

In the embodiment shown in FIGS. 4A-4C, the retaining component 107 is non-spherical, but is functionally similar to spherical retaining component embodiments in that it allows retention of the dried beads 105, in the vessel 104 without inhibiting the flow of the fluid 108 before and after the reaction occurs. The retaining component 107 may be shaped and sized to block the movement of the dried beads 105, through the opening 103 of the vessel 104, such as shown in FIG. 4C.

The retaining component 107 may comprise a central core or hub 109. The central core or hub 109 may define at least one passage 110 that permits the flow of fluid 108 through the passage 110. The passages 110 may allow any air bubbles beneath the retaining component 107 to pass through and exit the opening 103 of the vessel 104. For example, the addition of the fluid 108 may increase pressure beneath the retaining component 107. The passages 110 may also allow gases created through the reaction of the beads 105 to escape/be vented. The passages 110 are configured to minimise and avoid capture of the lyophilised test material. For example, the passages 110 may have smooth surfaces. The passages 110 may be sized to allow fluid to flow through in a substantially straight path. An amount of the fluid 108 exiting the passage 110 may be the same as an amount of the fluid 108 that entered the passage 110. An amount of reagent or primer in the fluid 108 may be unchanged as it passes through the passage 110.

As most clearly shown in FIG. 4C, the central core or hub 109 of the retaining component 107 may comprise a first end and a second end, wherein the second end may be disposed opposite the first end. The central core or hub 109 may comprise a first surface 111 at the first end and a second, or opposing surface 112 at the second end. The one or more passages 110 may extend between the first surface 111 and the second surface 112. In some embodiments, the first surface 111, and the second surface 112 are parallel.

Some or all of the passages 110 may generally have a straight path that extends directly between the surfaces 111, 112. The retaining component 107 may be arranged so that the first surface 111 faces the opening 103 of the vessel 104, and the second surface 112 faces the one or more dried beads 105 within the chamber of the vessel 104. Each one of the passages 110 may have a diameter that is smaller than a diameter of the one or more dried beads 105 to prevent the one or more dried beads 105 from entering (and moving through) the passage 110. Similarly, a diameter of the passages 110 may be smaller than a diameter of the mixing component 106. The diameter of the passages 110 may vary along the length of the passages. For example, the diameter of the passage 110 at the second surface 112 may be smaller than the diameter at the first surface 111.

The central core or hub 109 of the retaining component 107 may have a plurality of projecting portions 113 that extend outwardly from the central core or hub 109. The projecting portions 113 may extend outwardly in the same plane, such as parallel to the first and second surfaces 111, 112. The retaining component 107 may be substantially gear-shaped, wherein the projecting portions 113 resemble gear teeth. A recess 114 may be defined between adjacent ones of the projecting portions 113. Some or all of the projecting portions 113 may, together with the central core or hub 109, define some or all of the passages 110. Some or all of the passages 110 may be defined within some or all of the projecting portions 113.

The retaining component 107 may comprise a plurality of outwardly facing side surfaces, such as 113A and 114A as subsequently discussed. The projecting portions 113 may comprise contact surfaces 113A that are shaped to generally conform to the inner curvature of the vessel 104 to allow a close fit (or contact) with the inside of the vessel 104. The contact surfaces 113A may be convex. The contact surfaces 113A may engage the interior wall of the vessel 104 (defining the chamber) with a press fit to fix the retaining component 107 in the chamber. For example, to achieve a press fit, the outer diameter of the retaining component 107 may be slightly larger than the inner diameter of the vessel 104. Example diameters are subsequently disclosed herein. When the retaining component 107 is inserted into the vessel 104, it is done with enough force that the retaining component 107 is compressed and the vessel 104 expanded. This creates a seal between the two parts, holding them together without the need for additional fasters or adhesives.

An interior diameter of the chamber in which the retaining component 107 is received may be around 5 mm. The maximum diameter of the retaining component 107, as measured across outer surfaces 113, may be in the range of 4.5 mm to 5.2 mm. A smaller diameter, such as around 4.5 mm, would allow the retaining component 107 to fit loosely inside the 5 mm diameter of the vessel 104. A larger diameter, such as around 5.2 mm, would allow the retaining component 107 to fit more tightly inside the 5 mm diameter of the vessel 104, as a press/friction/interference fit. The diameters of the vessel 104 and the retaining component 107 would be paired according to the desired fit while ensuring that the gap with the vessel wall is smaller than the diameter of the dried bead 105.

The recesses 114 may include recessed surfaces 114A that are shaped to generally curve away from an inner wall of the vessel 104 and create a gap 115 (FIG. 4B) with the inside of the vessel 104. The recessed surfaces 114A may be concave, away from the interior wall of the vessel 104. The recesses 114 may work in combination with the passages 110 to facilitate the simultaneous flow of fluid 108 and gases past or through the retaining component 107. Some embodiments may have a plurality of the recesses 114. For example, fluid 108 may pass the retaining component 107 via some or all of the gaps 115, while gases (either trapped air or reaction gases) may pass through the retaining component 107 via the passages 110. The mixing component 106 may agitate the mixture to encourage gases or bubbles to move upwards and through the gaps 115 and/or through the passages 110. The inventors have identified that if these bubbles are not removed from the fluid 108, the bubbles can attach to the vessel wall or be caught at the base of the vessel 104. These bubbles interfere with the reaction and may also give optical reading errors.

The retaining component 107 may further comprise a prong 116 extending from the first surface 111 of the retaining component 107. The prong 116 may extend from a central portion of the central core or hub 109. The prong 116 provides a handle for holding the retaining component 107 to facilitate loading into (and removal from) the vessel 104. The prong 116 may be handled by a mechanical gripper or a vacuum assisted manipulator for manual or automated loading and/or removal.

In the described embodiments, the retaining component 107 is composed of one or more polymers or plastics. In some embodiments, the retaining component 107 is composed of a composite plastic. In the embodiment shown in FIGS. 3A-3C, the retaining component 107 is a 3.5 mm 0.9 g/cm3 sphere or ball composed of polypropylene (such as manufactured by Kaifeng Bell Stainless Steel Ball Manufacture Co., Ltd). Polypropylene is a thermoplastic polymer made of monomer propylene through chain growth polymerisation. Polypropylene is naturally white, mechanically strong, and resistant to many chemical solvents, including bases, acids alkali, alcohol, many inorganic substances, salt solutions, solvents, gasoline, and water. However, it will be apparent to those skilled in the art that in some other embodiments the retaining component 107 can be composed of any other suitable polymer material that does not absorb liquid and has good chemical resistance. Non-limiting examples of other suitable materials include polystyrene and acrylic.

In some embodiments, the retaining component 107 comprises a wax material or wax-based material. The wax may melt in the vessel 104 to form a lid or cap that floats on top of the fluid 108. The unmelted wax may take the form of a bead, similar to the spherical embodiment of the retaining component 107 discussed herein. The wax bead may retain the dried beads 105 in the vessel 104 while allowing fluid 108 to be added (which flows around the wax retainer bead 107).

As with the other embodiments of the retaining component 107 discussed herein, the wax retainer component 107 is positioned in the chamber between the dried beads 105, and the opening 103 to the vessel 104. When the fluid 108 is added to the chamber, the fluid 108 flows around (or through) the wax retainer component 107 to contact the dried beads 105. The fluid 108 can be added to fill the chamber until the dried beads 105 are immersed in the fluid 108. The wax retainer component 107 floats on the fluid 108.

The wax retainer component 107 may comprise beeswax, paraffin or a blend of waxes. Waxes may be selected according to their various properties, including their melting temperature, hardness, moulding features, and lack of interference with the reaction etc. The specific characteristics of the selected wax play a role in the operational performance of the retaining component 107.

To initiate or expedite the reaction between the test sample and the reconstituted compounds from the dried beads 105, heat may be introduced to the vessel 104. Upon reaching the amplification temperature, generally around 65° C., the wax constituting the retaining component 107 may begin to melt. This melting process might start as early as 55° C. and the wax would typically be entirely liquefied by the time the temperature reaches 65° C.

Once melted, the wax transitions into a liquid phase that floats on the surface of the fluid 108, which may be in a water buffer liquid phase. In doing so, it not only minimises the loss of reaction volume but potentially reduced the likelihood of false positives. The inventors have determined that false positives may be more likely when the concentration of the primers/probes increases at the evaporating surfaces of the reaction. Hence, the melted wax layer serves as a barrier against evaporation, thereby maintaining the reaction's integrity and enhancing the reliability of the results.

Ideally, the density of the chosen suitable material of the retaining component 107 is less than the density of water to enable it to float. However, in some embodiments, the retaining component 107 is in the form of a hollow shell to allow it to float even if the density of the shell is not less than that of water. The shell may define a hollow, wherein the shell may have at least one aperture or perforation. When the fluid 108 is added to the chamber, the fluid 108 flows around and/or through the hollow shell to contact the dried beads 105. The perforation is sized to avoid inadvertent filtering of the fluid 108. The amount of reagent or primer in the fluid is unchanged as it passes through the hollow shell. Optionally, the hollow of the retaining component 107 may be at least partly filled with a wax or wax-based material that may liquify and escape from the shell through the at least one aperture or perforation. The shell may be made from one or more polymers or plastics as discussed herein, such as polypropylene. The inclusion of wax within a hollow polypropylene shell may enhance manageability, considering polypropylene beads are less sticky than certain wax beads.

In some embodiments, the retaining component 107 is made of a hydrophobic material, and in other embodiments the retaining component 107 is made of a hydrophilic material. In some embodiments, the outer surface of the retaining component is functionalised with molecules, for example, proteins (e.g. enzymes), nucleic acids (e.g. probes) or catalysts, which assist with the testing of the sample.

The retaining component 107 can be of any practical size, providing that it is able to fit within the vessel 104, and form or allow a gap 115 (between the inner side wall of the vessel 104 and the retaining component 107) that is narrower than the size of the one or more dried beads 105, but sufficiently wide to allow the fluid 108 to flow through the gap 115 to reach the dried beads 105 in a practical timeframe. In some embodiments, the size of the gap 115 is greater than 1 mm but less than 2 mm in size. In some embodiments, the gap 115 is less than 1 mm. In some embodiments, the diameter of the retaining component 107 is 3.5 mm, 4 mm, 4.5 mm, 3 mm, and 2.5 mm. FIGS. 5A and 5B show examples of various sizes of the retaining component 107, using the spherical embodiment by way of example. The non-spherical embodiment of the retaining component 107 may also be sized to form or allow the same configuration of gap 115 to allow fluid to flow past or through. FIGS. 5C-5E show a smaller size of spherical retaining component 107 in different configurations of the vessel 104, as subsequently described herein. It will be apparent to those skilled in the art that the retainer component 107 can have any practical diameter provided that it is sufficiently large to prevent egress of the lyophilised beads 105, but not so large as to substantially restrict the addition of the fluid 108 to the vessel 104 and the dried beads 105 therein.

Referring again to FIG. 2, in the described embodiments the one or more dried beads 105 are approximately 1-3 mm in diameter. Preferably, the one or more dried beads 105 are lyophilised bead(s) or freeze-dried beads. The one or more dried beads 105 are mechanically deposited in the vessel 104 during the manufacturing process. These one or more dried beads 105 are composed of lyophilised materials, which in some embodiments include detection molecules (e.g., probes, quenchers and dyes), enzymes (e.g., DNA polymerase and reverse transcriptase), reagents (e.g., DNTPs or other PCR mix components). The lyophilised bead materials can be used for the purposes of nucleic acid amplification assays including, but not limited to, PCR and isothermal amplification of target nucleic acids from e.g. SARS-CoV-2, Influenza virus, Chlamydia, Gonorrhoea, HBV and HCV.

Conventionally, all required wet reagents for an assay e.g. primers, fluorescent probes, enzymes, dNTPs) are combined and lyophilises together as a single unit, such as a cake or a bead. Consequently, the single units of beads or cake are homogenous, or composed of the same materials. A problem with this approach is that the reagents can sometimes prematurely interact with one another, for example, during storage or before being used to test for a specific analyte or group of analytes.

In the described embodiments, the one or more dried beads 105 may comprise beads that are individually configured to respond to specific analytes. At least one or more or the dried beads 105 may be made from materials that differ from the rest. In other words, some of the dried beads 105 are heterogenous or distinct in type or kind. For example, a first dried bead may respond to a first analyte, and a second dried bead may react to a second analyte but not the first analyte. Having individual beads composed of different materials, and that react to different analytes, reduces the likelihood of premature reactions occurring before the bead reagent interacts with the analyte. For example, some beads may combine a plurality of reagents prior to lyophilisation, which may over time interact over time, leading to false-positive interactions. This is substantially mitigated by separating the individual reagents into separate beads. Each of the dried beads 105 may comprise an excipient and/or betaine. The excipient and the betaine may help prevent undesired reactions and ensure the stability of the dried bead 105, as discussed in further detail below.

During the manufacture, assembly and storage before use, the various kinds of types of dried beads can be kept separate. The inventors have found that separating the dried bead reagents significantly improves inventory efficiency and shelf life in storage, compared to a configuration where bead reagents and other bead components are mixed prior to lyophilisation. For example, each of the bead reagents can be stored as individual beads, and a mixture of beads chosen when required—i.e. “made to order” when the specific combination of beads is desired to test for a particular analyte or group of analytes. Storing the bead reagent as a bead occupies minimal space compared to storing lyophilised materials in vials and tubes.

The inventors have found that lyophilising beads is more efficient than lyophilising a larger volume of liquid in a tube. This is because the smaller volume and spherical nature of the bead allows for more efficient sublimation of the water out of the frozen material during the primary drying phase of lyophilisation.

Individual reagent beads can be subject to quality control (QC) measures, having to dispose of an entire bead comprised of combined reagents due to a QC failure in a single reagent. The inventors have observed that this methodology facilitates the assembly process and minimises wastage costs. For example, in the context of producing varying configurations of diagnostic cartridges, only one or two beads need to be replaced out of an average of 6 or 7 beads, to target a different test or pathogen. QC tests may be implemented for each lyophilised bead, using methods such as visual inspection or automated machine vision. The dried beads 105 may be made to contain very precise amounts of reagent; for example, to within 0.1 μL. A real-time digital imaging QC system may be employed to ensure that each dried bead 105 meets the specified volume requirement.

QC tests may consist of multiple assessments, including a crush test to assess physical integrity, residual water content, and a functional test within an assembled assay. The QC test may include counting the dried beads 105 and/or checking the shape/size of each dried bead 105 (to ensure it does not pass through the gap between the retaining component 107 and the vessel wall). The QC test may comprise checking dried bead 105 colours if visual dyes are used to colour the beads.

The one or more dried beads 105 may comprise a first lyophilised primer bead configured to interact with a first gene target/target nucleic acid. A subsequent reaction is indicative of the presence of that target (analyte). The one or more dried beads 105 may contain one or more polymerase chain reaction reagents and oligonucleotide primers. The first lyophilised primer bead may, for example, be a set of primers for a SARS-CoV-2 target sequence or some other gene target.

The one or more dried beads 105 may comprise a second lyophilised primer bead configured to interact with a second gene target/target nucleic acid, with the subsequent reaction indicating the presence of that target (analyte). The second lyophilised primer bead may target a second gene of interest, for example, influenza target sequence. In some embodiments, the second lyophilised primer bead is a control target (e.g. human housekeeping gene). In some embodiments, the one or more dried beads 105 comprises two or more lyophilised primer beads. The two or more lyophilised primer beads may comprise the first lyophilised primer bead and the second lyophilised primer bead. In some embodiments, the one or more dried beads 105 comprises three or more lyophilised primer beads. The three or more lyophilised primer beads may comprise the first lyophilised primer bead, the second lyophilised primer bead, and a third lyophilised primer bead. The third lyophilised primer bead may be configured to interact with a third gene target/target nucleic acid that is different to the targets of the first and second lyophilised primer beads. The number of lyophilised primer beads may be chosen based on the number of targets to be detected. In some embodiments, the third lyophilised primer bead is configured to interact with the same target as one of the first or second lyophilised primer beads.

The separation of different primers into distinct beads reduces the likelihood of unwanted interactions among various primer sets. The inventors have identified that separating the primers into separate beads enables the manufacture of different types of tests using different combinations of primer beads. For example, a first test “Test A” may comprise the use of the first primer bead and the second primer bead. Further, a second test “Test B” may comprise the use of the first primer bead and the third primer bead, and so on. This flexibility allows for the creation of varying combinations of primer beads to meet differing requirements, streamlining manufacturing efficiencies with respect to bead quantities needed to meet test demand. For example, both Test A and Test B require the first primer bead, so more of these beads can be manufactured, thereby reducing the need for excessive manufacture of the second and third primer beads.

Each of the dried beads 105 may comprise an excipient in addition to the primer. The excipient is a reagent that protects the main compound of that bead (e.g. the primer) from freeze damage during the lyophilisation process. Moreover, these excipients give physical structure and integrity to the lyophilised bead, enabling its physical handling and loading into a cartridge. The excipients are often polysaccharides that contribute to the final viscosity of the reaction mixture. Suitable excipients for this purpose may include, but limited to, trehalose, sucrose, and dextran.

Each of the dried beads 105 may comprise reagents like betaine. Betaine may be added into the primer beads only. Betaine facilitates a chemical interaction that prevents non-specific primer-primer interactions (commonly referred to as “primer-dimers”). The inventors have found that primer-dimers significantly contribute to false positive interactions in nucleic acid amplification assays. The inclusion of betaine in a separate primer bead allows for higher concentration of the betaine, thereby increasing its effectiveness in preventing primer-primer interactions. The inventors have identified that a lower concentration of betaine in the final combined reaction enhances the interaction between primers and the target. A primer bead with high betaine concentration effectively prevents non-specific interactions, however, when all the reagents are combined, the betaine concentration is reduced thereby allowing efficient amplification in the final reconstituted reaction.

The one or more dried beads 105 may comprise a lyophilised probe quencher bead. The probe quencher bead is configured to detect the amplified genes using a fluorescent probe and a quencher, following the same principles as real-time PCR. As with the primers, the probes are also gene target specific. The probe quencher bead may also contain excipients and betaine for the reasons outlined above. In some embodiments, separate probe-quencher beads may be used for different gene targets. In some embodiments, the probe quencher beads are combined for different targets.

The one or more dried beads 105 may comprise a lyophilised enzyme bead. In some embodiments, the lyophilised enzyme bead comprises a DNA polymerase that drives the nucleic acid amplification reaction. In the case of a RNA target, this bead may also include a reverse transcriptase enzyme. The lyophilised enzyme bead may also include excipients but typically would not include betaine because there is not a primer-primer interaction issue for this bead. One advantage of having a separate protein enzyme bead is that it physically separates the active site of the polymerase from primers, target gene segments, dNTPs and other assay components like magnesium etc. Some enzymes are formulated with antibodies or aptamers into the function pocket to stop non-specific amplification. Typically, these ‘hot start’ proteins require heat to displace the antibody or aptamer from the functional pocket, allowing the functional domains to be open and available for amplification. The inventors have identified that a separate enzyme bead serves a similar function to a ‘hot start’ enzyme, preventing non-specific interaction with the functional site of the polymerase by compartmentalising them in separate beads. In some assays, more polymerase is required for efficient performance. This requirement can be met by adding more enzyme into the bead or by including an additional enzyme beads. This configuration offers flexibility to titrate the amount of the reagent required in the final reaction.

The one or more dried beads 105 may comprise a lyophilised deoxynucleotide triphosphate (dNTP) bead. The dNTP bead is a key building-block of nucleic acid amplification. In some embodiments, the one or more dried beads 105 comprises a lyophilised control bead that provide a means for users to assess proper functioning of the reaction. The control bead may comprise an exogenous control target, whereby the lack of reaction or amplification of this exogenous control target may indicate that the reaction is not occurring properly. For example, an embodiment of the container or apparatus 100 may be used for testing water samples to identify faecal contamination of a water source. The faecal contaminated water sample also includes inhibitors that obstruct the reaction, which could result in a ‘false negative’ test result. To prevent this, the apparatus 100 may include an exogenous control bead (e.g. containing a target comprising genes from an extinct dodo) and primers designed to amplify the exogenous control target. If the amplified exogenous control target remains undetected upon the anticipated completion of the reaction, the test is deemed invalid rather than negative. One advantage of using an exogenous control target (such as genes from an extinct dodo) is that if the reaction does occur, these genes are unlikely to be confused with the actual target genes.

The magnetic mixing component 106 facilitates mixing when the fluid 108 is added to the vessel 104, leading to the dissolution and reconstitution of the active components within the dried beads 105. The magnetic mixing component 106 can be made from any suitable magnetic material; for example, stainless steel or iron. In the described embodiment, the magnetic mixing component 106 is a spherical bead made from a magnetic stainless steel alloy, for example, a 440 grade stainless steel may be used. In one embodiment, the steel magnetic mixing component 106 is 2 mm in diameter, for example, 2 mm Diameter Grade 100 Hardened AISI 420 Stainless Steel Ball Bearing (Simply Bearings). In another embodiment, the steel magnetic mixing component 106 is 4 mm in diameter. It will be apparent to those skilled in the art that the magnetic mixing component 106 can be any size or range of sizes provided that it is compatible with the dimensions of the vessel 104 and the one or more dried beads 105. The magnetic mixing component 106 may be sized to be similar in size to most or all of the beads 105.

The magnetic mixing component 106 may be sized so that it complements the shape and/or size of the vessel 104. In some embodiments, the vessel 104 has a rounded end (such as a test tube) wherein the mixing and reaction of the beads 105 with the sample occurs. The magnetic mixing component 106 may be a ball or sphere with a diameter that is slightly smaller than or equal to the diameter of the rounded end. This similarity in diameters allows the magnetic mixing component 106 to sit snugly within the curvature of the rounded end. If the diameter of the magnetic mixing component 106 is larger than the diameter of the rounded end, the magnetic mixing component 106 contacts the walls of the vessel 104 and is prevented from contacting the very bottom of the vessel 104. This creates a pocket of space under the magnetic mixing component 106 in which undissolved reagents might accumulate. Furthermore, with all else being equal, a smaller-sized mixing component 106 is able to move around within the vessel 104 more than a larger-sized mixing component 106, leading to more efficient and/or more even mixing of the fluid in the chamber of the vessel 104.

By way of example, a container or apparatus 100 may comprise a vessel 104 with a volume of 200 μL. The vessel 104 may comprise lyophilised beads 105, each of which may contain about 4 μL of solid material (dried compounds). The vessel 104 may have a straight tube section which defines an opening at one end, and at the opposite end of the tube section the vessel 104 may have a tapered lower section with a rounded tip. The vessel 104 contains retaining bead 107. The retaining bead 107 may be sized to stay within the straight tube section and be too large to enter the tapered lower section, such as shown in FIG. 2. The retaining bead 107 may rest against the walls of the vessel 104 at or around the entry to the tapered lower section, thereby being spaced away from the rounded tip of the vessel 104 so that the magnetic mixing component 106 has room to move around. In embodiments such as shown in FIG. 3C, the retaining bead 107 may enter the tapered lower section but still be too large to reach the rounded tip of the vessel 104. The retaining bead 107 may rest partway along the tapered lower section, such as about halfway along, and thereby be spaced away from the rounded tip of the vessel 104. A volume of 100 μL of fluid 108 (containing the sample) may be added to the vessel 104. The fluid 108 dissolves the beads 105 and releases the compounds. The mixing bead 106, which is sized to sit in the rounded tip of the vessel 104, agitates the fluid 108 to ensure the dissolved compounds are adequately mixed to react with the sample. The total volume after mixing may be about 120 μL. In a 200 μL vessel, the fluid level may ascend to about halfway along the straight section of the tube. The test may allow for higher reaction volumes than standard PCR reactions, which typically occupy volumes in the range of 25 μL or less.

In some embodiments, one or more magnetic mixing components 106 are added to the apparatus or container 100 for testing of a biological or environmental sample. When the apparatus or container 100 is placed within an instrument that generates a movable magnetic field, this causes the magnetic mixing component 106 within the apparatus or container 100 to move correspondingly, and thereby provide mixing. During the mixing process, the retaining component 107 can float and move away from the magnetic mixing component 106, facilitating undistributed mixing.

Referring to FIG. 2 and FIGS. 5A-5E, the container 100, configured for testing biological or environmental samples, comprises a vessel 104 that can be manufactured separately to, or integrally formed with, a cap 102. The vessel 104 may be in the form of a tube, can be of various sizes and shapes and may be injection moulded or otherwise formed from a rigid plastic or any other suitable material. For example, in some embodiments, the tube 104 is a 200 μL polypropylene PCR tube (PCR-02-NC or equivalent). In various other embodiments, the tube is a 1.5 mL screw cap or flip-top cap tube (FIGS. 5C and 5D) or a smaller PCR tubes (FIG. 5E). The screw cap may comprise cap threads (not shown) that are configured to engage with a corresponding thread (not shown) on the vessel 104.

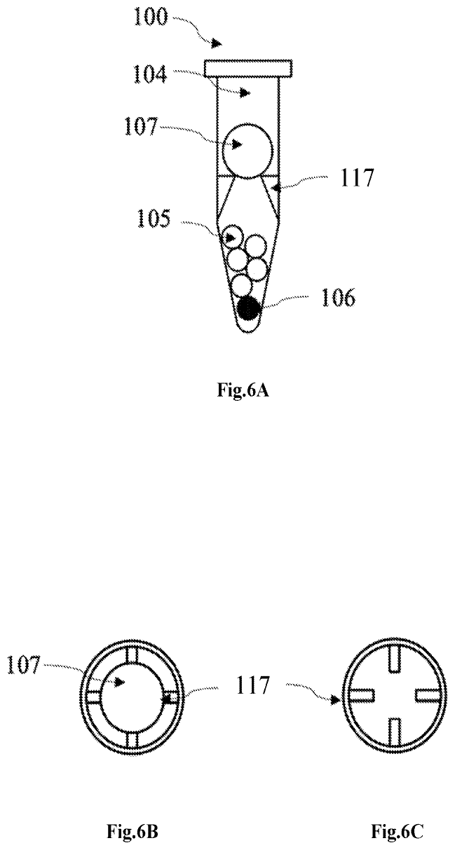

In some embodiments, the tube or vessel 104 is configured to support the retaining component 107 and prevent it from falling to the bottom of vessel component 104. For example, in the embodiment of FIGS. 6A and 6B, the tube 104 includes features or projections 117 extending from the inner tube surface of the tube 104 towards its centre to support the retaining component 107. FIG. 6C shows the projections 117 without the retaining component 107.

In some embodiments, a 96-well plate formed of small PCR tubes is used. Referring to FIG. 7, the container or apparatus 100 may be provided in a cartridge 101 which holds one or more of the apparatuses or containers 100. The vessel 104 and cap 102 can be made of any suitable material known to those skilled in the art. The material should not interfere with the analysis or measurement of the test sample. In the illustrated embodiment, the vessel 104 is transparent and the cap 102 is opaque. In another embodiment, the cap 102 is also transparent. The vessel 104 may be transparent to provide visibility of the one or more dried bead(s) 105 when disposed in the chamber. The vessel 104 may be transparent to provide visibility of the sample when received in the chamber. The vessel 104 may be transparent to provide visibility of the retaining component 107 when received in the chamber.

### Method for Using a Tube Containing a Spherical Retainer

FIG. 2 illustrates the method 200 for using a container or apparatus 100 for testing of a biological or environmental sample. In some embodiments, the method 200 comprises steps 201, 202, and 203, as discussed in more detail below.

As discussed herein, the container or apparatus 100 comprises a vessel 104 containing a retaining component 107 and one or more dried beads 105 in accordance with an embodiment of the present disclosure. The vessel 104 may comprise a straight tube section which defines an opening at one end that is covered by a cap 102. At the opposite end of the tube section the vessel 104 may have a rounded tip. The rounded tip may be end part of a lower section which tapers from the tube section. The tube section may contain a fluid/fill line, which is used to indicate the appropriate amount/level of the fluid 108 to be added to the vessel 104. The fluid/fill line may be about halfway along the tube section.

In the context of the embodiments described herein, the process starts with a vessel 104 containing one or more dried beads 105 and a retaining component 107 of an embodiment of the present disclosure. The user opens the vessel 104 by removing the cap 102 (at 201). Upon removing the cap 102, the one or more dried beads 105 remain within the upright vessel 104 due to the weight of the retaining component 107 applying a downward force to the one or more dried beads 105, and the size of the retaining component 107 being such that the one or more dried beads 105 cannot fit between the gap made by the inner side wall of the vessel 104 and the retaining component 107.

After removal of the cap 102 at 201, at 202 the user then adds fluid 108 to the vessel 104, which flows around or otherwise past the retaining component 107 and through the gap to reach the one or more dried beads 105 at the bottom of the vessel 104. As the fluid 108 contacts the one or more dried beads 105, they are dissolved by the fluid 108. Once all of the fluid has been added, the retaining component 107 can float above the fluid line.

In some embodiments, the container or apparatus 100 for testing of a sample additionally contains a magnetic mixing component 106, preferably in the form of a steel mixing bead. The container or apparatus 100 is configured to be placed within an instrument that generates a movable magnetic field that causes the magnetic mixing component 106 within the vessel 104, preferably a tube, to move correspondingly, and thereby provide mixing of the vessel's contents (at 203). The cap 102 may be refitted after the biological or environmental sample is added to the vessel 104 for testing. The cap 102 may stop biological material escaping (or contaminants entering) as the test is running. The magnetic field may be generated by an external permanent magnet that is moved in proximity of the vessel 104 to move the magnetic mixing component 106.

The magnet may be moved close to the upper region of the vessel 104, causing the magnetic mixing component 106 to rise through the reaction fluid towards the magnet. The magnetic mixing component 106 will then drop under the gravity when the magnet is moved away. An oscillating mechanism can be used to move the magnet, causing the magnetic mixing component 106 to repeatedly rise and fall in response to the fluctuating magnetic field, thereby inducing mixing of the fluid in the vessel 104. Alternatively, an electromagnet consisting of an electrical coil and an optional pole piece can be used to cause a directed magnetic field to move or lift the magnetic mixing component 106 when the electromagnet is energised, and then allow the magnetic mixing component 106 to drop when the electromagnet is de-energised. In some embodiments, the external magnetic field can actively lift the magnetic mixing component 106 and pull it back down to an alternative position or move the magnetic mixing component 106 horizontally from side to side to induce mixing. The reading of the test result may be by a colorimetric, fluorescence or bioluminescent instrument. If the sample test uses fluorescent detection in the tube or cartridge, then non fluorescent visual dyes can be used and configured to not interfere with the sample test or the subsequent fluorescent optical test detection. In this arrangement, the required visual or fluorescent optical measurements required to read the test result are restricted to the lower region of the vessel 104. The sequencing of the mixing mechanism (comprising the magnet) is then synchronised to lift the magnetic mixing component 106 up or clear of the required result reading region in the test fluid 108 during optical measurements. The magnetic mixing component 106 may also move the retaining component 107 where additional clearance is required for optical measurements such as tests with low fluid volumes.

Once the reaction has been completed, the reading of the reaction mixture is facilitated by the magnetic field moving the magnetic mixing component 106 out of the way. For example, the reading of the sample may occur within the tapered lower section of the vessel 104, so the magnetic field may lift the magnetic mixing component 106 out of the tapered lower section to the top of the fluid line. The retaining component 107 is also lifted by the magnetic mixing component 106, which provides an upward force on the retaining component 107. This provides clear and unobstructed access for the reading of the sample at the bottom of the vessel 104. In other embodiments, the magnetic mixing component 106 is disposed above the retaining component 107 and by applying a magnetic field to the vessel 104, both the magnetic mixing component 106 and the retaining component 107 are brought to the bottom of the vessel 104, providing a clear and unobstructed access for the reading of the sample at the top of the vessel 104.

### Kits

The apparatus or container 100 for testing a biological or environmental sample described herein may also be provided as a component of a test kit.

In the case of a kit for use in detecting a nucleic acid in a sample, the kit can include additional reagents, for example, lysis buffer or buffer for preparing the sample, and additional apparatuses or containers for diluting the sample.

Optionally, a kit of the present disclosure is packaged with instructions for use in a method described herein.

As used herein “kit” means a collection of apparatus and reagents for performing an assay or test. A typical test kit for such tests includes a small box containing separate apparatus and reagents that a user individually opens and uses. Typically, such a test kit will contain a test tube or moulded consumable and separate powder and liquid reagents, along with a separate package that contains the test strip or cartridge.

The reagents may be supplied separately from the test kit, and are dispensed by the user into the tubes following sampling. Only once the user has inserted the swab or sample into the liquid reagents do they then introduce the prepared sample to the tube. There is considerable error that may be introduced during this process by the user, and there is a time delay as the sample is washed in the reagents and introduced to the tube. The described kits address this problem by providing a tube or vessel which is pre-filled with the test reagents in the form of lyophilised (freeze dried) beads (e.g., primers for tests). The user simply adds the sample to the test tube containing the lyophilised beads.

A kit may be “complete”, where all reagents needed for preparation and running of the test are provided. Alternatively, a kit may be “partial”, omitting certain reagents needed for operation. Both complete and partial kits may include additional reagents for sample preparation such as nucleic acid isolation.

### EXAMPLES

The present disclosure includes the following non-limiting examples.

Tube containing spherical retainer for use in COVID-19 ZiP-CoVx-P2 point-of-care test.

Coronaviruses are a large family of RNA viruses which can cause disease in animals and humans. SARS-CoV-2 is a betacoronavirus that was first reported in Wuhan, Hubei Province, China, and has since rapidly spread globally. The virus causes COVID-19 (coronavirus disease 2019). Infection may be asymptomatic or may cause mild to lethal clinical manifestations. Those most at risk for developing severe illness are the elderly, immunocompromised, and those with pre-existing medical conditions such as hypertension, diabetes, or respiratory and cardiovascular disease.

SARS-CoV-2 transmission occurs through aerosol, droplet, or surface contact. High numbers of asymptomatic and mild cases unknowingly transmit the infection. Identification of such individuals requires a high sensitivity testing method, such as nucleic acid amplification. Rapid and accurate molecular testing is required for successful clinical management and transmission control of symptomatic and asymptomatic SARS-CoV-2 infection.

SARS-CoV-2 virus is generally detectable in upper respiratory specimens during the acute phase of infection. Positive results are indicative of the presence of RNA from SARS-CoV-2 virus. A positive result does not rule out possible co-infection with other pathogens. A positive test result does not necessarily imply that SARS-CoV-2 infection is the cause of the presenting disease, and must be interpreted in the context of the clinical presentation and broader epidemiological context. Positive results must be reported to the appropriate health authorities in accordance with local reporting requirements, and is the responsibility of the user. Negative results do not preclude SARS-CoV-2 infection and should not be used as the sole basis for patient management decisions. Negative results must be combined with clinical observations, patient history, and epidemiological information.

The ZiP-CoVx-P2 point-of-care test with the ZiP-P2 instrument (as described at www.zipdiag.com/zip-test-systems) enables decentralisation and point-of-care diagnosis of SARS-CoV-2 by utilising isothermal nucleic acid amplification technology. The ZiP-CoVx-P2 diagnostic system utilises a NAT assay-based technology for detection of SARS-CoV-2 RNA. The test provides a high-sensitivity result that is rapid (<40 minutes from sample input to result output), simple to use, robust, and offers automated result interpretation and data capture. This technology employs novel primer design, highly efficient nucleic acid amplification, and fluorescent probes to facilitate high sensitivity and high specificity detection.

The function of the ZiP-CoVx-P2 test is to aid diagnosis of COVID-19 in symptomatic individuals, or to screen for SARS-CoV-2 infection in asymptomatic individuals. The test is intended for use in dedicated test spaces (e.g., hospital emergency, intensive care, general practice, antiviral treatment clinics, or other sites established for screening and testing purposes). The test can also be used by laboratory-trained professionals in pathology settings. Minimal training is required as the test is menu-driven with a screen-prompted automated workflow that includes result interpretation and reporting. Training includes reading the Instructions for Use and following the screen-prompted workflow.

A synthetic flocked swab is used to obtain an oropharyngeal (throat) and bilateral mid-turbinate (nasal) sample. Dry swab samples must be used because swabs in liquid transport media may interfere with test performance. The test allows for two alternative sample collection workflows including local swab sampling acquired near the testing site and remote swab sampling where transport of the swab is required. The local swab workflow proceeds by which the patient sample is added directly to the lysis tube and the test is run immediately. Alternatively, the remote swab workflow, where immediate testing is not possible, the swab is stable for 72 hours at 2° C. to 30° C.

SARS-CoV-2 RNA amplification and detection reagents, as well as those for a human internal control, are provided as ready-to-use dried beads or lyophilised beads in two sealed reaction tubes containing respective retaining components that are configured together in the ZiP-CoVx-P2 Test Cartridge. Each tube has a different SARS-CoV-2 gene target—M or Orf1b—and a human gene target—RNaseP internal control. Addition of the processed patient sample reconstitutes the lyophilised beads. The Test Cartridge is then loaded into the ZiP-P2 instrument where amplification of the target nucleic acid sequence occurs and is detected.

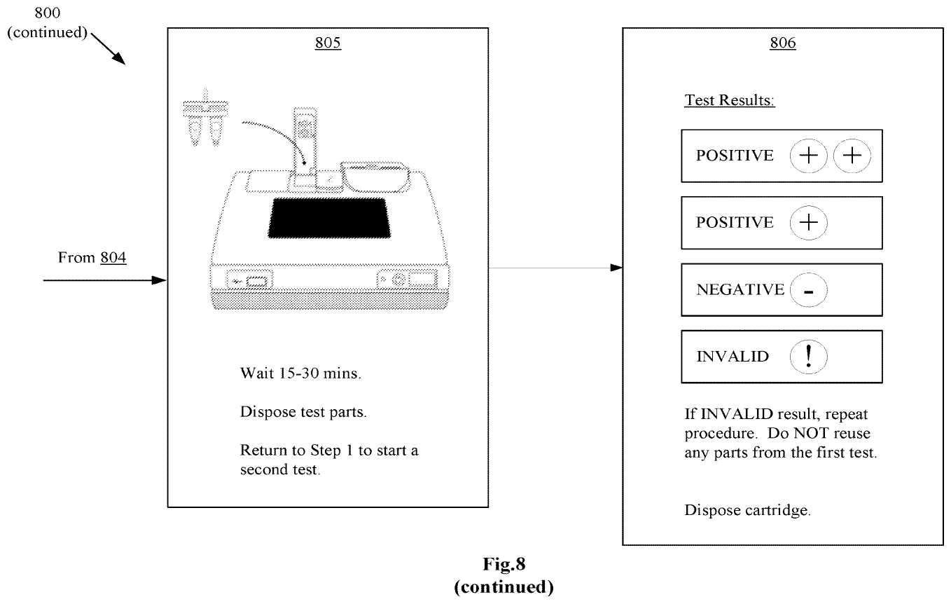

Embodiments of a container or apparatus 100 for testing of a sample, for example, the apparatus or container of FIGS. 7 and 8, can be used in COVID-19 point of care testing. FIG. 8 provides a series of images outlining the use of a container or apparatus 100 for testing of a biological or environmental sample of the present disclosure.

An example test procedure or method 800 involves first placing the sample preparation tray onto the instrument sample preparation deck and loading the test parts into their respective positions on the tray, as shown at 801. The test parts consist of Tube 1 (containing 1 L of ZIP lysis buffer), and Tube 2 (containing 900 μL of ZIP lysis buffer and a 2 mm steel ball for instrument-mediated fluid mixing) (tubes are polypropylene tubes, Type Axygen SCT-200-C-S or equivalent). The P2 cartridge contains two 200 μL polypropylene PCR tubes (PCR-02-NC or equivalent) connected by a cartridge carrier with a tag. Each PCR tube contains a 3.5 mm polypropylene (0.90 g/cm3) ball/sphere for retaining beaded lyophilised material (FIG. 7(1)). Additionally, each tube contains SARS-CoV-2 test and endogenous control lyophilised assay material (including primers) as 6 separate beads, which can be reconstituted through the addition of lysis buffer and sample solution. Additionally, each cartridge tube contains a 2 mm steel ball (magnetic bead) for instrument-mediated fluid mixing.

The user waits 5 minutes for Tube 1 to preheat, and then at 802, swirls the patient swab 10 times in Tube 1 and waits a further 5 minutes for heating. At 803, 100 μL of sample is transferred from Tube 1 to Tube 2 using a provided pipette, and the instrument mediates fluid mixing via the steel mixing ball for 30 seconds. The person skilled in the art will appreciate the many different arrangements of volumes that may be used in alternative test configurations.

The sample in Tube 2 is then transferred to each of the P2 cartridge tubes (Tube A and B), which contain the one or more dried beads 105, the magnetic mixing component 106 and the retaining component 107 of the present disclosure, as shown at 804. The P2 cartridge is closed by folding over the attached cap and pressing down firmly. At 805, the P2 cartridge is placed into the instrument to run the test, and the results are available after 15-30 minutes. Target amplification signals within the valid range and endpoints defined by a minimum control determine whether the amplification signals are considered detectable. After the test is complete, the P2 cartridge tubes are thrown away. A second test may be run by repeating the method 800, starting from 801. At 806, the potential test results are shown. The potential test results provided include SARS-CoV-2 target nucleic acids (M and Orf1b) are detected in the sample (POSITIVE “++”), a SARS-CoV-2 target nucleic acid (M or Orf1b) is detected in the sample for one nucleic acid targets (POSITIVE “+”), neither of the SARS-CoV-2 target nucleic acids are detected in the sample (NEGATIVE “−”) and the presence or absence of SARS-CoV-2 nucleic acids in the sample cannot be determined (INVALID “!”). If the result is invalid, the test procedure should be repeated by repeating the method 800, starting from 801. None of the parts from the first test, such as the P2 cartridge tubes, should be reused.

Once inside the reader, there is transcription of RNA into cDNA and isothermal amplification of that cDNA. The primers included in each of the P2 cartridge tubes will recognise a different conserved region of SARS-CoV-2, and can be read using a fluorescence detector within the machine.

Many modifications will be apparent to those skilled in the art without departing from the scope of the present disclosure.

1. An apparatus for testing of a biological or environmental sample, the apparatus including:
a vessel defining an interior chamber and an opening to the chamber, with the opening being the only means of entry and exit from the chamber;
one or more dried beads disposed within the chamber, and wherein the opening is configured to allow a fluid containing the biological or environmental sample to enter the chamber for testing of a reaction with the one or more dried beads therein; and
a retaining component disposed between the opening and the one or more dried beads within the chamber; and
wherein the retaining component is configured to prevent the one or more dried beads from exiting the vessel through the opening when open, and to allow fluid in the vessel to flow past and/or through the retaining component to interact with the one or more dried beads therein.

2. The apparatus of claim 1, wherein the vessel is transparent to facilitate visualisation of at least one of: (i) the one or more dried beads; (ii) the sample; and/or (iii) the retaining component when disposed within the chamber.

3. The apparatus of claim 1 or claim 2, wherein the one or more dried beads include: (i) diagnostic reagents; (ii) one or more polymerase chain reaction reagents; and/or (iii) one or more oligonucleotide primers.

4. The apparatus of any one claims 1 to 3, wherein the one or more dried beads comprises a first lyophilised primer bead containing a first lyophilised primer, wherein the first lyophilised primer is configured to be reconstituted by the fluid to interact with a first target nucleic acid.

5. The apparatus of claim 4 when dependent on claim 3, wherein the first lyophilised primer bead comprises at least one of the one or more polymerase chain reaction reagents.

6. The apparatus of any one of claims 1 to 5, wherein the one or more dried beads comprises a second lyophilised primer bead containing a second lyophilised primer, wherein the second lyophilised primer is configured to be reconstituted by the fluid to interact with a second target nucleic acid.

7. The apparatus of any one of claims 1 to 6, wherein the one or more dried beads comprises three or more lyophilised primer beads.

8. The apparatus of any one of claims 1 to 7, wherein the one or more dried beads comprises: (i) a lyophilised probe quencher bead; (ii) a lyophilised enzyme bead; and/or (iii) a lyophilised deoxynucleotide triphosphate (dNTP) bead.

9. The apparatus of any one of claims 3 to 8, wherein each of the lyophilised beads comprises: (i) an excipient; and/or (ii) betaine.

10. The apparatus of any one of claims 1 to 9, further including a magnetic mixing component.

11. The apparatus of claim 10, wherein the magnetic mixing component is composed of steel.

12. The apparatus of any one of claims 1 to 11, wherein the retaining component is approximately spherical.

13. The apparatus of any one of claims 1 to 12, wherein the retaining component comprises a hub defining at least one passage extending between opposing first and second surfaces of the retaining component and configured to allow the fluid to enter and exit the retaining component through the at least one passage, wherein the first surface faces the opening of the vessel, and the second surface faces the one or more dried beads within the chamber of the vessel.

14. The apparatus of claim 13, wherein a diameter of the at least one passage is smaller than a diameter of the one or more dried beads to prevent the one or more dried beads from passing through the passage.

15. The apparatus of claim 13 or claim 14, wherein the retaining component comprises a plurality of outwardly facing side surfaces, and at least one recessed surface, wherein the recessed surface is recessed relative to at least one of the outwardly facing side surfaces so as to be positioned away from an interior wall of the vessel.

16. The apparatus of claim 15, wherein the plurality of outwardly facing side surfaces comprises a contact surface configured to engage the interior wall of the vessel with a press fit to secure the retaining component within the chamber.

17. The apparatus of any one of claims 13 to 16, wherein the retaining component comprises a prong extending from the first surface of the retaining component.

18. The apparatus of any one of claims 1 to 17, wherein the retaining component is composed of: (i) a composite plastic; (ii) polypropylene; (iii) polystyrene; (iv) a wax material; or (v) a wax-based material.

19. The apparatus of any one of claims 1 to 18, wherein the retaining component is a polypropylene sphere comprising a shell defining: (i) a hollow; and (ii) at least one aperture in the shell in communication with the hollow, wherein the aperture is configured to allow the fluid to enter and exit the retaining component through the shell.

20. The apparatus of claim 19, wherein the hollow is at least partly filled with a wax or wax-based material.

21. The apparatus of any one of claims 1 to 20, wherein the retaining component is not a filter.

22. The apparatus of any one of claims 4 to 21 when dependent on any one of claims 3 to 9, wherein an amount of reagent or primer in the fluid is unchanged as it passes through or around the retaining component during interaction with the sample in the fluid.

23. The apparatus of any one of claims 13 to 17 or claim 19, wherein the retaining component is configured for an amount of the fluid exiting the retaining component to be the same as an amount of the fluid that entered the retaining component.

24. The apparatus of any one of claims 1 to 23, wherein the retaining component is configured to: (i) float on top of the fluid; (ii) be partly immersed in the fluid; or (iii) be fully immersed in the fluid.

25. A diagnostic test kit including:
the apparatus of any one of the preceding claims, and
lysis buffer.

26. A diagnostic test system comprising:
the diagnostic test kit of claims 25; and
a test instrument configured to generate a movable magnetic field, wherein movement of the magnetic field causes a corresponding movement of a magnetic mixing component within the apparatus to mix contents within a vessel of the apparatus.

27. A test method, including:
introducing a fluid containing a biological or environmental sample into the apparatus of any one of the preceding claims.

28. The test method of claim 27, wherein the apparatus includes a magnetic mixing component, and the method further includes moving the magnetic mixing component to the top of the apparatus to facilitate reading of a test result.

29. The test method of claim 28, wherein the reading of the test result is by a colorimetric, fluorescence or bioluminescent instrument.

30. The test method of claim 28 or claim 29, comprising applying a movable magnetic field to the magnetic mixing component, wherein movement of the movable magnetic field causes corresponding movement of the magnetic mixing component within the vessel of the apparatus.

31. A method of manufacturing lyophilised beads, wherein the lyophilised beads are subjected to quality control (QC) tests comprising at least one of:
(i) counting the beads;
(ii) checking sizes of the beads; and
(iii) checking bead colours if visual dyes are used to colour the beads.
